(19) United States
(12) Patent Application Publication
(10) Pub. No.: US 2025/0322941 A1
(43) Pub. Date: Oct. 16, 2025

(54) MEDICAL SERVICE SUPPORT DEVICE, MEDICAL SERVICE SUPPORT METHOD, AND MEDICAL SERVICE SUPPORT PROGRAM

(71) Applicant: FUJIFILM Corporation, Tokyo (JP)

(72) Inventors: Yosuke OHASHI, Tokyo (JP); Kota KAWAGUCHI, Tokyo (JP); Jun SHINGU, Tokyo (JP); Yuki WADA, Tokyo (JP)

(73) Assignee: FUJIFILM Corporation, Tokyo (JP)

(21) Appl. No.: 19/172,554

(22) Filed: Apr. 7, 2025

(30) Foreign Application Priority Data
Apr. 11, 2024 (JP) 2024-064206

(51) Int. Cl. G16H 40/20

(52) U.S. Cl. CPC G16H 40/20

(57) ABSTRACT

A processor acquires medical practice information including date and time related to a medical practice and information related to an index value related to the medical practice, derives a difference between a first statistical value of the information related to the index value in the comparison target period and a first statistical value of the information related to the index value in the reference target period for each of a plurality of item values of at least one item, derives an influence degree on the increase or decrease in the index value in the reference target period relative to the comparison target period for each item value of the item, based on a second statistical value based on the medical practice information in the reference target period and the difference, and performs a display process for displaying the influence degree for at least one item value included in the item.

### CROSS REFERENCE TO RELATED APPLICATIONS

The present application claims priority from Japanese Patent Application No. 2024-064206, filed on Apr. 11, 2024, the entire disclosure of which is incorporated herein by reference.

### BACKGROUND

### Technical Field

The present disclosure relates to a medical service support device, a medical service support method, and a medical service support program.

### Related Art

In medical institutions, various medical practices such as medical interviews, diagnoses, examinations, treatments, and nursing care are performed on patients. Various methods for analyzing a result of such a medical practice have been proposed. For example, JP2023-106605A proposes a method of providing a determination reason for a failure (referred to as an imaging failure) in a case of capturing a radiation image and information for correcting the imaging failure, in a case of performing an examination using the radiation image.

Although the imaging failure occurs for various reasons, the method disclosed in JP2023-106605A provides an individual determination reason for the imaging failure. Therefore, in a case of considering a certain period such as a month, it is not possible to statistically identify what reason or attribute is a main cause of the occurrence of the imaging failure in the period.

### SUMMARY OF THE INVENTION

The present disclosure has been made in view of the above circumstances, and an object of the present disclosure is to identify a statistical factor that affects a result of a medical practice.

A medical service support device according to the present disclosure comprises: at least one processor, in which the processor is configured to acquire medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value, derive a difference between a first statistical value of the information related to the index value in the comparison target period and a first statistical value of the information related to the index value in the reference target period for each of a plurality of item values of at least one predetermined item, derive an influence degree on the increase or decrease in the index value in the reference target period relative to the comparison target period for each item value of the at least one item, based on a second statistical value based on the medical practice information in the reference target period and the difference, and perform a display process for displaying the influence degree for at least one item value included in the at least one item.

A medical service support method according to the present disclosure comprises: acquiring medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value; deriving a difference between a first statistical value of the information related to the index value in the comparison target period and a first statistical value of the information related to the index value in the reference target period for each of a plurality of item values of at least one predetermined item; deriving an influence degree on the increase or decrease in the index value in the reference target period relative to the comparison target period for each item value of the at least one item, based on a second statistical value based on the medical practice information in the reference target period and the difference; and performing a display process for displaying the influence degree for at least one item value included in the at least one item.

A medical service support program according to the present disclosure causes a computer to execute: a procedure of acquiring medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value; a procedure of deriving a difference between a first statistical value of the information related to the index value in the comparison target period and a first statistical value of the information related to the index value in the reference target period for each of a plurality of item values of at least one predetermined item; a procedure of deriving an influence degree on the increase or decrease in the index value in the reference target period relative to the comparison target period for each item value of the at least one item, based on a second statistical value based on the medical practice information in the reference target period and the difference; and a procedure of performing a display process for displaying the influence degree for at least one item value included in the at least one item.

According to the present disclosure, it is possible to identify a statistical factor that affects a result of a medical practice.

### BRIEF DESCRIPTION OF THE DRAWINGS

FIG. 1 is a perspective view showing an outline of a medical information system to which a medical service support device according to an embodiment of the present disclosure is applied.

FIG. 2 is a diagram showing medical practice information.

FIG. 3 is a diagram showing a hardware configuration of the medical service support device according to the embodiment of the present disclosure.

FIG. 4 is a diagram showing a functional configuration of the medical service support device according to the embodiment of the present disclosure.

FIG. 5 is a diagram showing a display screen.

FIG. 6 is a diagram showing a display screen.

FIG. 7 is a diagram showing a display screen.

FIG. 8 is a flowchart showing processing performed in the present embodiment.

FIG. 9 is a diagram showing another example of display of an influence degree display region on the display screen.

FIG. 10 is a diagram for describing correction of an influence degree.

FIG. 11 is a diagram showing another example of display of a main screen.

FIG. 12 is a perspective view showing another outline of the medical information system to which the medical service support device according to the embodiment of the present disclosure is applied.

FIG. 13 is a diagram showing another example of display of the main screen.

### DETAILED DESCRIPTION

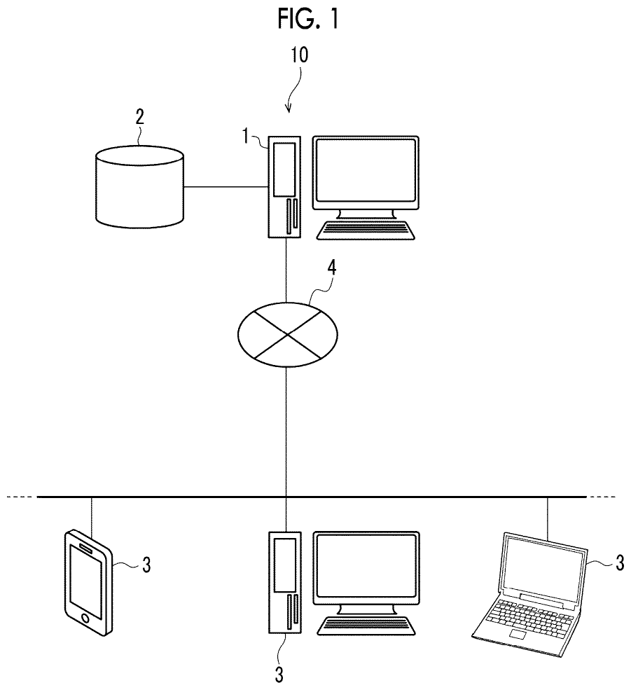

Hereinafter, an embodiment of the present disclosure will be described with reference to the drawings. First, a configuration of a medical service support system to which a medical service support device according to an embodiment of the present disclosure is applied will be described. FIG. 1 is a diagram showing a schematic configuration of the medical service support system. In a medical service support system 10 shown in FIG. 1, a medical service support device 1, a management server 2, and a plurality of client terminals 3 are connected to each other via a network 4 in a communicable manner. The medical service support system 10 is a system used for at least one medical institution (for example, a hospital) that handles a plurality of pieces of medical information.

The medical service support device 1 performs a process of analyzing an influence on an increase or decrease in an index value related to a medical practice of a medical worker for each predetermined item value based on information acquired from the management server 2, as described below. In the present embodiment, the medical service support device 1 performs a process of analyzing an influence on an increase or decrease in an imaging failure rate for each predetermined analysis value, with respect to an imaging failure, which is a failure in capturing a medical image. A detailed configuration of the medical service support device 1 will be described below.

The management server 2 includes a server computer or the like that manages various types of information in the hospital transmitted from the client terminal 3. The management server 2 may be installed in a medical institution or may be a cloud server installed on a cloud. In the present embodiment, the management server 2 manages medical practice information related to a medical practice of the medical worker. In the present disclosure, the medical worker is not limited to qualified persons such as a doctor, a technician, a pharmacist, and a nurse, and also includes in-hospital staff who performs reception of medical checkup or examination, guidance of patients, and the like, or unqualified persons who assist qualified persons. The management server 2 may manage not only the medical practice information transmitted from one medical institution but also the medical practice information transmitted from a plurality of medical institutions.

The client terminal 3 is a terminal device owned by the medical worker in the medical institution. Examples of the client terminal 3 include a workstation, a personal computer, a tablet terminal, a smartphone, and a smartwatch. In addition, a console of an imaging apparatus such as an X-ray imaging apparatus, a computed tomography (CT) apparatus, and a magnetic resonance imaging (MRI) apparatus installed in the medical institution may be used.

Examples of the network 4 include a wide area network (WAN). The WAN is merely an example, and the network 4 may be composed of at least one of a local area network (LAN), a WAN, or the like.

In the client terminal 3, information related to the medical practice of the medical worker is managed. The information related to the medical practice includes an index value related to the medical practice. The information on the medical practice including the index value is managed in the client terminal 3 in association with a date and time of the medical practice. Further, the information related to the medical practice managed by the client terminal 3 is transmitted to the management server 2 together with the date and time and is managed as medical practice information.

In the present embodiment, as the information managed in the client terminal 3, for example, in a case of the client terminal 3 of a doctor, information such as a doctor name, a medical department, a patient name, a patient gender, a patient date of birth, a medical checkup date and time, a disease name, an examination content, an examination result, a diagnosis content, a treatment content, a treatment result, a content of surgery, a result of surgery, whether the patient is hospitalized or outpatient, a ward of the patient in a case of hospitalization, a reception time, and a report creation time is included. In addition, in a case of the client terminal 3 of an examination room, as the information managed in the client terminal 3, an examination type, an examination room, an examination device, an examination date and time, a technician name, a patient name, a patient gender, a patient date of birth, whether the patient is hospitalized an outpatient, a ward of the patient in a case of hospitalization, an examination reception time, an examination start time, an examination end time, a waiting time, a requesting department, a requesting doctor, an examination reservation time, examination reservation information (whether it is on the day or whether it is an emergency), an examination start time, an examination end time, a time when the examination implementation is input to the client terminal 3, a time when an examination report is created, an examination state, a pathology request, and the like are included. In particular, in a case in which the examination room performs an examination involving the capture of a medical image, as the information managed in the client terminal 3, a type of a modality (a radiography apparatus, a CT apparatus, an MRI apparatus, an ultrasound imaging apparatus, a fluoroscopy apparatus, mammography, an endoscopy apparatus, an in vitro diagnostic apparatus, and the like) in a case of capturing a medical image, an imaging part, an imaging menu, an imaging condition, presence or absence of imaging failure, a patient waiting time, an imaging preparation time, a time required for imaging required, an image adjustment time, an examination report creation time, a cleaning time of an apparatus, an index value based on an acquired image, and the like are included.

Here, the imaging condition includes an irradiation dose, an exposure dose, a dose index (EI: exposure index, DI: deviation index), a tube voltage, an mAs value, and the like in a case of radiography. The index value based on the image includes a noise amount and sensitivity (S value) of the image.

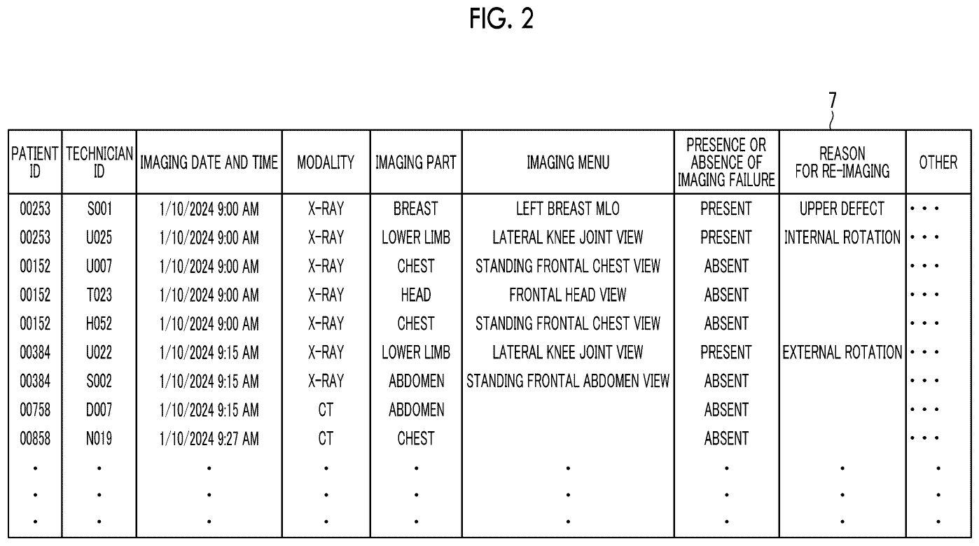

The management server 2 converts the information transmitted from the client terminal 3 into a database to manage the medical practice information related to the medical practice of the medical worker in one medical practice unit. FIG. 2 is a diagram showing an example of a database of the medical practice information managed by the management server 2. In the present embodiment, since the medical service support device 1 provides an influence degree of an increase and decrease in the imaging failure to the medical institution, a database 7 of the medical practice information shown in FIG. 2 includes information for deriving the influence degree of the increase and decrease in the imaging failure, such as an imaging menu and the presence or absence of the imaging failure.

As shown in FIG. 2, the database 7 of the medical practice information includes a patient ID, a technician ID, an imaging date and time, a modality, an imaging part, an imaging menu, the presence or absence of the imaging failure, a reason for re-imaging, and other information in one practice information unit. The modality is a type of the imaging apparatus, and includes an X-ray imaging apparatus, a CT apparatus, an MRI apparatus, an ultrasound imaging apparatus, and the like. The imaging part is a part to be a target for acquiring a medical image, and includes a breast, a lower limb, a chest, a head, an abdomen, and the like. The imaging menu is a menu for defining a technique and the like in a case of performing imaging according to the modality, and in a case of the X-ray imaging apparatus, the imaging menu includes a left breast MLO, a frontal chest view, a lateral knee joint view, a frontal head view, and the like. The presence or absence of the imaging failure includes information of “Present” in a case in which the imaging has failed and the imaging failure has occurred, and includes information of “Absent” in a case in which the imaging has succeeded and the imaging failure has not occurred. The reason for re-imaging is a reason for the imaging failure and the need for re-imaging.

In capturing an X image, the imaging failure occurs due to reasons such as a positioning error of the patient (upper defect, internal rotation, external rotation), body movement of the patient, insufficient inhalation, a setting error of an imaging condition, or detection of a foreign object.

In addition, in the management server 2, the database 7 of the medical practice information may be managed for each medical institution. A plurality of times of imaging may be performed on the same patient in one examination. In such a case, the management server 2 may compile the medical practice information for each examination. In such a case, the database 7 of the medical practice information may include the number of times of imaging and the number of imaging failures in one examination.

In a case of performing imaging, the date and time related to the medical practice is an imaging date and time, but may be at least one of a start date and time of the medical practice, an end date and time of the medical practice, a reservation date and time of the medical practice, a reception date and time of the medical practice, or a call date and time for implementation of the medical practice.

The medical service support device 1 may be a workstation or a personal computer installed in a hospital or may be a server computer installed on a cloud. The medical service support device 1 has a medical service support program according to the present embodiment installed therein. The medical service support program is stored in a storage device of another server computer connected to the network or a network storage (neither shown) in a state of being accessible from an outside, and is downloaded and installed in the medical service support device 1 upon request. Alternatively, the medical service support program is distributed in a state of being recorded on a recording medium such as a digital versatile disc (DVD) or a compact disc read only memory (CD-ROM), and is installed in the medical service support device 1 from the recording medium.

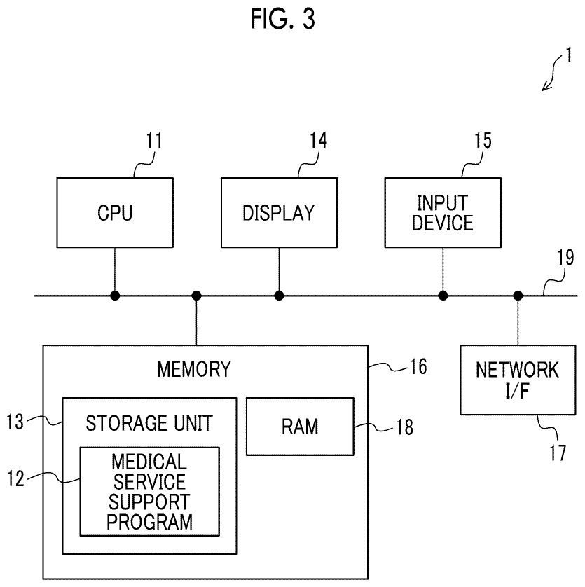

FIG. 3 is a diagram showing a hardware configuration of the medical service support device according to the present embodiment. As shown in FIG. 3, the medical service support device 1 includes a central processing unit (CPU) 11, a display 14, an input device 15, a memory 16, and a network interface (I/F) 17 connected to the network 4. The CPU 11, the display 14, the input device 15, the memory 16, and the network I/F 17 are connected to a bus 19. The CPU 11 is an example of a processor in the present disclosure.

The memory 16 includes a storage unit 13 and a random access memory (RAM) 18. The RAM 18 is a memory for primary storage and is, for example, a RAM such as a static random access memory (SRAM) or a dynamic random access memory (DRAM).

The storage unit 13 is a non-volatile memory, and is implemented by at least one of, for example, a hard disk drive (HDD), a solid state drive (SSD), an electrically erasable and programmable read only memory (EEPROM), or a flash memory. The storage unit 13 as a storage medium stores a medical service support program 12 according to the present embodiment. The CPU 11 reads out the medical service support program 12 from the storage unit 13, loads the medical service support program 12 into the RAM 18, and executes the loaded medical service support program 12.

The display 14 is a device that displays various screens and is, for example, a liquid crystal display or an electro luminescence (EL) display. The input device 15 is a device for a user to provide input and is, for example, at least any of a keyboard, a mouse, a microphone for voice input, a touchpad for proximity input including a contact, or a camera for gesture input. The network I/F 17 is an interface for connecting to the network 4.

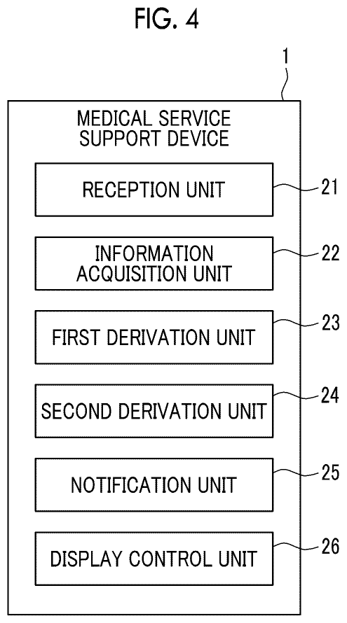

Next, a functional configuration of the medical service support device according to the present embodiment will be described. FIG. 4 is a diagram showing a functional configuration of the medical service support device according to the present embodiment. As shown in FIG. 4, the medical service support device 1 comprises a reception unit 21, an information acquisition unit 22, a first derivation unit 23, a second derivation unit 24, a notification unit 25, and a display control unit 26. Then, the CPU 11 executes the medical service support program 12, so that the CPU 11 functions as the reception unit 21, the information acquisition unit 22, the first derivation unit 23, the second derivation unit 24, the notification unit 25, and the display control unit 26.

In the present embodiment, the medical service support device 1 performs a process of analyzing an influence on an increase or decrease in the index value related to the medical practice of the medical worker for each predetermined item value. Specifically, the medical service support device 1 performs a process of analyzing an influence on an increase or decrease in an imaging failure rate for each of a plurality of item values of at least one predetermined item, with respect to an imaging failure, which is a failure in capturing a medical image. The imaging failure rate is an example of an index value in the present disclosure.

The reception unit 21 receives an analysis instruction from the client terminal 3 of the medical institution. In this case, the reception unit 21 receives designation of a period for analysis input by an operator via the client terminal 3 of the medical institution that has received the analysis instruction. Specifically, the reception unit 21 receives designation of a reference target period T1, which is a target period for analysis of the increase or decrease in the imaging failure rate, and a comparison target period T2, which is a target period for comparison of the increase or decrease in the imaging failure rate, input by the operator via the client terminal 3.

The reference target period T1 and the comparison target period T2 may have the same length or different lengths. For example, the reference target period T1 may be March and the comparison target period T2 may be February, or the reference target period T1 may be April and the comparison target period T2 may be January to March. In addition, a start date and time of the reference target period T1 may be before a start date and time of the comparison target period T2. For example, the reference target period T1 may be from April 1 to April 30, and the comparison target period T2 may be from March 1 to March 31. Conversely, the start date and time of the reference target period T1 may be after the start date and time of the comparison target period T2. For example, the reference target period T1 may be from March 1 to March 31, and the comparison target period T2 may be from April 1 to April 30.

The information acquisition unit 22 acquires medical practice information in which the date and time of the medical practice is included in the reference target period T1 and the comparison target period T2, from the management server 2. For example, in a case in which the reference target period T1 is from Mar. 1, 2024 to Mar. 31, 2024 and the comparison target period T2 is from Feb. 1, 2024 to Feb. 28, 2024, the information acquisition unit 22 acquires medical practice information with the medical practice dates and times from Mar. 1, 2024 to Mar. 31, 2024 and Feb. 1, 2024 to Feb. 28, 2024, from the management server 2.

The first derivation unit 23 derives, for each of a plurality of item values of at least one predetermined item, a difference between a first statistical value of information related to the index value in the comparison target period T2 and a first statistical value of information related to the index value in the reference target period T1, based on the medical practice information acquired by the information acquisition unit 22. In the present embodiment, the index value is an imaging failure, and the first statistical value is an imaging failure rate for each item value. The first statistical value may be an average value, a median value, a total value, a minimum value, or a maximum value for each item value instead of the imaging failure rate for each item value. The first statistical value can be derived as an average value or the like only in a case in which the information related to the index value is a numerical value. Here, as will be described below, information necessary for deriving the imaging failure rate is the presence or absence of the imaging failure and the number of times of imaging. The presence or absence of the imaging failure is included in the medical practice information, and the number of times of imaging in each of the reference target period T1 and the comparison target period T2 can be counted from the medical practice information. The presence or absence of the imaging failure and the number of times of imaging counted from the medical practice information are examples of the information related to the index value related to the medical practice.

Here, the medical practice information includes an imaging menu. The imaging menu is determined for each imaging part, and includes, for example, a plurality of menus such as a left breast MLO, a standing frontal chest view, a lateral knee joint view, and a frontal head view. The imaging menu is an example of an item in the present disclosure, and each menu included in the imaging menu is an example of an item value in the present disclosure. In the present disclosure, the item value includes text, but may include a numerical value such as an imaging date and time.

The medical practice information includes information on the presence or absence of the imaging failure for each medical practice as shown in FIG. 2. The first derivation unit 23 derives the imaging failure rate for each menu as a reference first statistical value R1 by dividing the total number of medical practices in which the imaging failure is “Present” for each menu by the number of times of imaging for each menu, for the reference target period T1. In addition, the first derivation unit 23 derives the imaging failure rate for each menu as a comparison first statistical value R2 by dividing the total number of medical practices in which the imaging failure is “Present” for each menu by the number of times of imaging for each menu, for the comparison target period T2. The reference first statistical value R1 and the comparison first statistical value R2 may be a decimal or a percentage.

The first derivation unit 23 further derives a difference RS by subtracting the comparison first statistical value R2 from the reference first statistical value R1 for each menu included in the imaging menu. For example, in a case in which the reference first statistical value R1 is 30% and the comparison first statistical value R2 is 20%, the difference RS is +10%, and, in a case in which the reference first statistical value R1 is 5% and the comparison first statistical value R2 is 10%, the difference RS is −5%.

The second derivation unit 24 derives an influence degree A0 on the increase or decrease in the index value in the reference target period T1 relative to the comparison target period T2 for each item value of at least one item, based on a second statistical value based on the medical practice information in the reference target period T1 and the difference RS. In the present embodiment, the influence degree A0 is derived by multiplying the second statistical value by the difference RS.

In the present embodiment, the second statistical value is a total value of the number of times of imaging for each menu included in the medical practice information. Therefore, the second derivation unit 24 calculates the total value of the number of times of imaging for each menu included in the imaging menu, and multiplies the calculated total value by the difference RS to derive the influence degree A0 on the increase or decrease in the imaging failure in the reference target period T1 relative to the comparison target period T2. For example, in a case in which the difference RS is +5% and the total value of the number of times of imaging is 100 for a certain menu, the influence degree A0 is derived as +5.0.

The notification unit 25 notifies the client terminal 3 of the derivation of the influence degree A0 via the second derivation unit 24. For the notification, any method such as an e-mail, a short message service (SMS), a telephone, or notification via an application can be used. The notification may simply notify that the derivation of the influence degree A0 via the second derivation unit 24 has been completed, or may notify of a result of the derivation of the influence degree A0.

The notification unit 25 may perform the notification in a case in which the influence degree A0 is derived by the second derivation unit 24, but may perform the notification only in a case in which the derived influence degree A0 satisfies a predetermined condition. For example, the notification may be performed in a case in which the number of menus of the influence degree A0 exceeding a predetermined threshold value Th1 exceeds a predetermined threshold value Th2. In addition, a standard deviation σ of the influence degree A0 may be derived, and the notification may be performed in a case in which the influence degree A0 exceeds, for example, 3σ. In addition, the notification may be performed in a case in which the number of times the influence degree A0 for each menu of the imaging menu exceeds a predetermined threshold value Th3 exceeds an upper limit.

The display control unit 26 performs a display process for displaying the influence degree A0 derived by the second derivation unit 24 on the client terminal 3. The display process may be either transmitting information necessary for displaying the influence degree A0 on the client terminal 3 to the client terminal 3, or generating a display screen for displaying the influence degree A0 and transmitting the generated display screen to the client terminal 3.

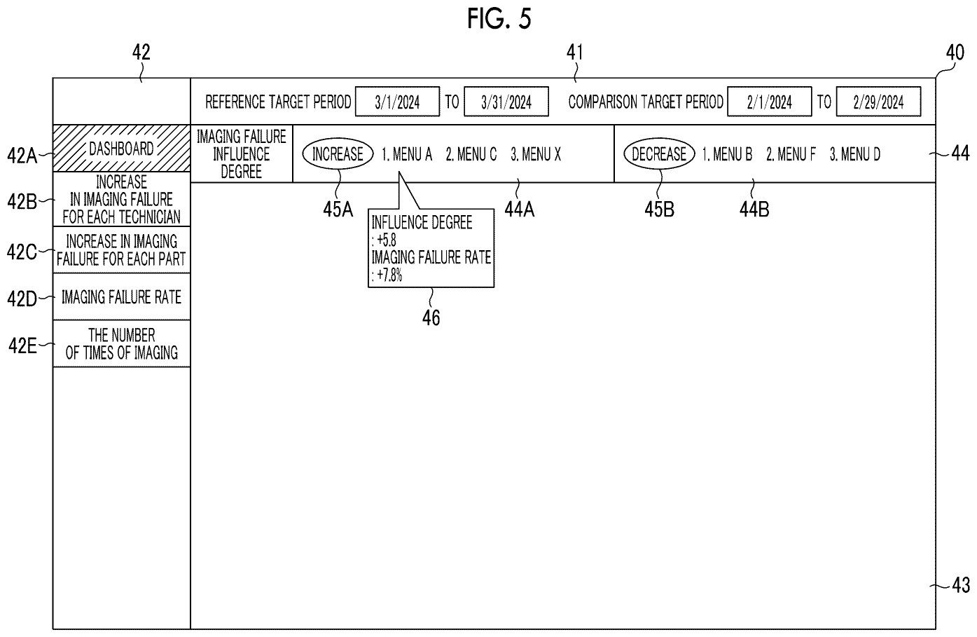

FIG. 5 is a diagram showing a display screen of the influence degree displayed on the client terminal 3. As shown in FIG. 5, a display screen 40 includes a period region 41, a selection region 42, and a main region 43.

The period region 41 is a region in which the reference target period T1 and the comparison target period T2 designated by the operator are displayed.

The selection region 42 includes an item 42A of “dashboard”, an item 42B of “increase in imaging failure for each technician”, an item 42C of “increase in imaging failure for each part”, an item 42D of “imaging failure rate”, and an item 42E of “the number of times of imaging”. In a case in which the operator selects the item 42A of the “dashboard” of the selection region 42, an influence degree region 44 is displayed in the main region 43. In FIG. 5, a menu region 44A for displaying the top three menus (menus A, C, and X) with the highest influence degree A0 among the imaging menus and a menu region 44B for displaying the bottom three menus (menus B, F, and D) with the lowest influence degree A0 are displayed in the influence degree region 44. An “increase” mark 45A is provided in the menu region 44A, and a “decrease” mark 45B is provided in the menu region 44B. As a result, it is found that, in the imaging using the menu displayed in the menu region 44A, the influence degree on the increase in the imaging failure is greater than in the comparison target period T2, and in the imaging using the menu displayed in the menu region 44B, the influence degree on the decrease in the imaging failure is greater than in the comparison target period T2.

In addition, in the influence degree region 44, the display of the menu is selected using a mouse cursor or the like, thereby displaying the influence degree A0 and the imaging failure rate for the selected menu using a pop-up 46. In FIG. 5, the menu A of the menu region 44A is selected, and the influence degree (+5.8) and the imaging failure rate (+7.8%) for the menu A are displayed by the pop-up 46.

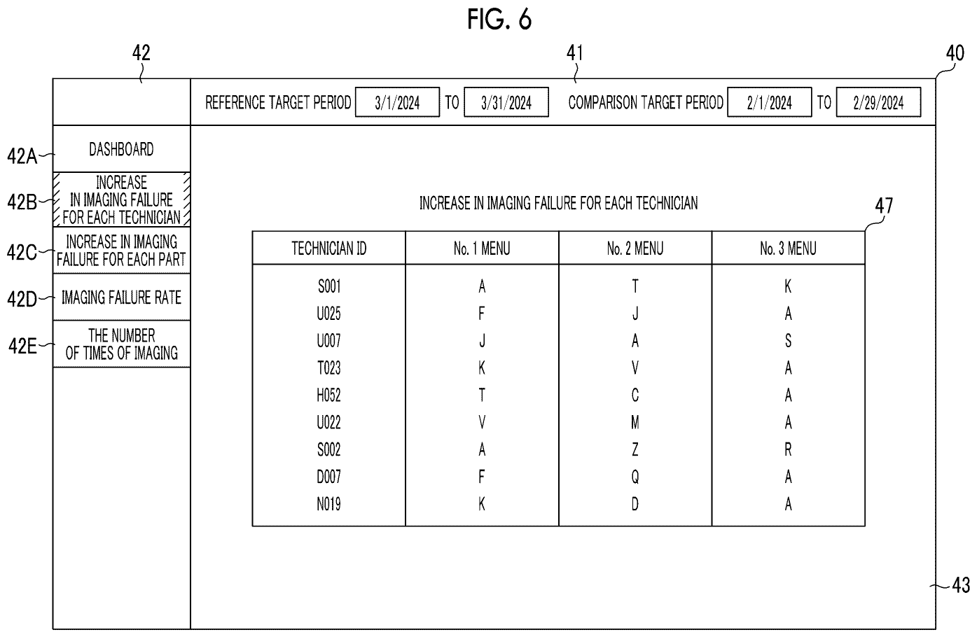

In addition, in a case in which the operator selects the item 42B of “increase in imaging failure for each technician”, as shown in FIG. 6, the increase in the imaging failure for each technician is displayed as a table 47 in the main region 43. The table 47 displays a technician ID and the top three menus with the highest influence degree on the increase in the imaging failure for a technician with the technician ID as a NO. 1 menu, a NO. 2 menu, and a NO. 3 menu. Although not shown, in a case in which the operator selects the item 42C of “increase in imaging failure for each part”, the increase in the imaging failure for each part is displayed as a table in the main region 43.

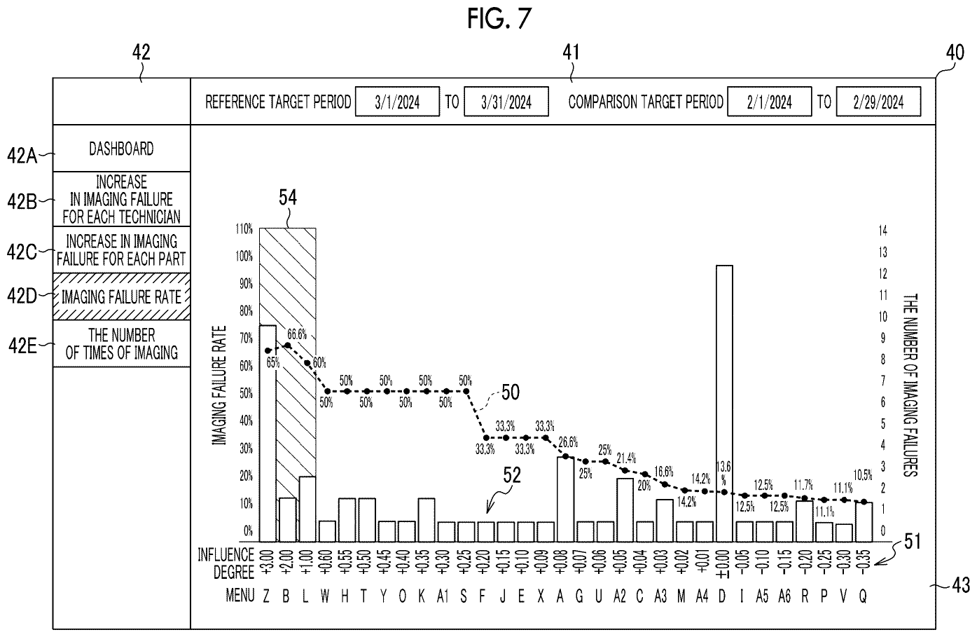

In addition, in a case in which the operator selects the item 42D of “imaging failure rate”, as shown in FIG. 7, a line graph 50 of the imaging failure rate is displayed in the main region 43. In the line graph 50, a vertical axis represents the imaging failure rate, and a horizontal axis represents individual menus of the imaging menu arranged from left to right in descending order of the influence degree. An influence degree 51 of the imaging failure derived for each menu is displayed between the graph and the display of the menu. In addition to the line graph 50 of the imaging failure rate, a bar graph 52 representing the number of imaging failures is displayed on the display screen 40 shown in FIG. 7. In addition, a diagonal line mark 54 is assigned to the top three menus with the greatest influence degree. The mark 54 may be assigned as a specific color instead of the diagonal line. A mark may be assigned to the bottom three menus with the smallest influence degree. In addition, although not shown, in a case in which the operator selects the item 42E of “the number of times of imaging”, a graph of the number of times of imaging is displayed in the main region 43.

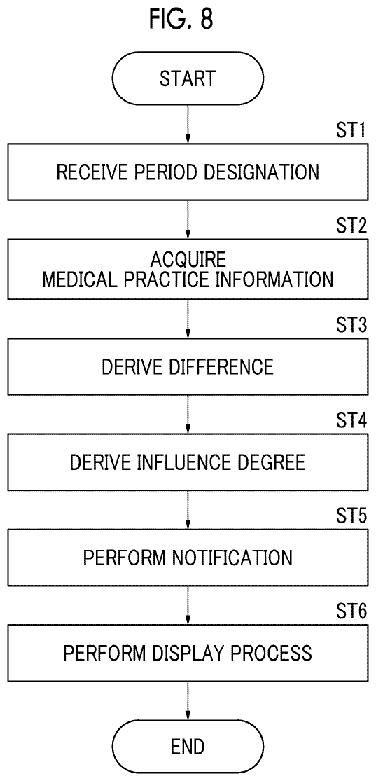

Next, processing performed in the present embodiment will be described. FIG. 8 is a flowchart showing the processing performed in the present embodiment. The processing is started in a case in which the operator issues an instruction from the client terminal 3, and the reception unit 21 receives the designation of the reference target period T1 and the comparison target period T2 input from the client terminal 3 by the operator (step ST1). Next, the information acquisition unit 22 acquires the medical practice information in which the date and time is included in the reference target period T1 and the comparison target period T2 from the management server 2 (step ST2). Subsequently, the first derivation unit 23 derives, for each of a plurality of item values of at least one predetermined item, the difference RS between the comparison first statistical value R2 of the index value in the comparison target period T2 and the reference first statistical value R1 of the index value in the reference target period T1, based on the medical practice information acquired by the information acquisition unit 22 (difference derivation; step ST3).

Next, the second derivation unit 24 derives the influence degree A0 on the increase or decrease in the index value in the reference target period T1 relative to the comparison target period T2 for each item value of at least one item, based on the second statistical value based on the medical practice information in the reference target period T1 and the difference RS (step ST4). Subsequently, the notification unit 25 notifies the client terminal 3 that the influence degree A0 has been derived (step ST5). Further, the display control unit 26 performs a display process for displaying the influence degree A0 on the client terminal 3 (step ST6), and the processing is ended.

As described above, in the present embodiment, the influence degree on the increase or decrease in the index value is derived for each of a plurality of item values of at least one predetermined item, and the influence degree is displayed for each item value of at least one item. Therefore, it is possible to identify which item value statistically affects the index value depending on the magnitude of the influence degree A0, and as a result, it is possible to specify a statistical factor that affects the increase or decrease in the index value.

In the above-described embodiment, the influence degree for each menu included in the imaging menu is displayed on the display screen 40, but the present invention is not limited thereto. The influence degree may be displayed only for one menu with the highest influence degree or only for one menu with the lowest influence degree. In addition, regarding the influence degree of the increase in the imaging failure for each technician shown in FIG. 6, the influence degree of the increase in the imaging failure may be displayed not only for a plurality of technicians but also for only one technician.

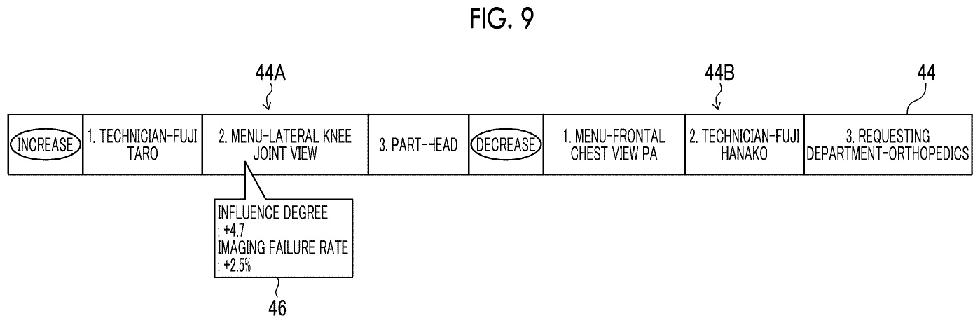

In addition, in the above-described embodiment, the influence degree is derived for one item, that is, for each menu of the imaging menu, but the present invention is not limited thereto. For example, the influence degree may be derived for each of a plurality of item values for each of a plurality of items. For example, in addition to or instead of the above-described imaging menu, the influence degree for each technician, the influence degree for each imaging part, the influence degree for each requesting department that requests imaging, and the like may be derived. In this case, for example, as shown in FIG. 9, the top three and bottom three items of the influence degrees derived for all of the imaging menu, the technician, the imaging part, and the requesting department need only be displayed in the influence degree region 44 of the display screen 40. Even in this case, in the influence degree region 44, the display of the menu is selected using the mouse cursor or the like, thereby displaying the influence degree A0 and the imaging failure rate for the selected menu using the pop-up 46. In FIG. 9, “2. Menu-Lateral knee joint view” in the menu region 44A is selected, and the influence degree (+4.7) and the imaging failure rate (+2.5%) for the selected menu are displayed by the pop-up 46.

In addition, in the above-described embodiment, the derived influence degree may be corrected based on a distribution of the first statistical value in a period longer than the reference target period T1 or the comparison target period T2. For example, an average and a standard deviation of the imaging failure rate for the past five months may be derived as the distribution of the first statistical value, a distance of the standard deviation σ from the average may be derived, and the influence degree derived for the reference target period T1 may be corrected based on the distance.

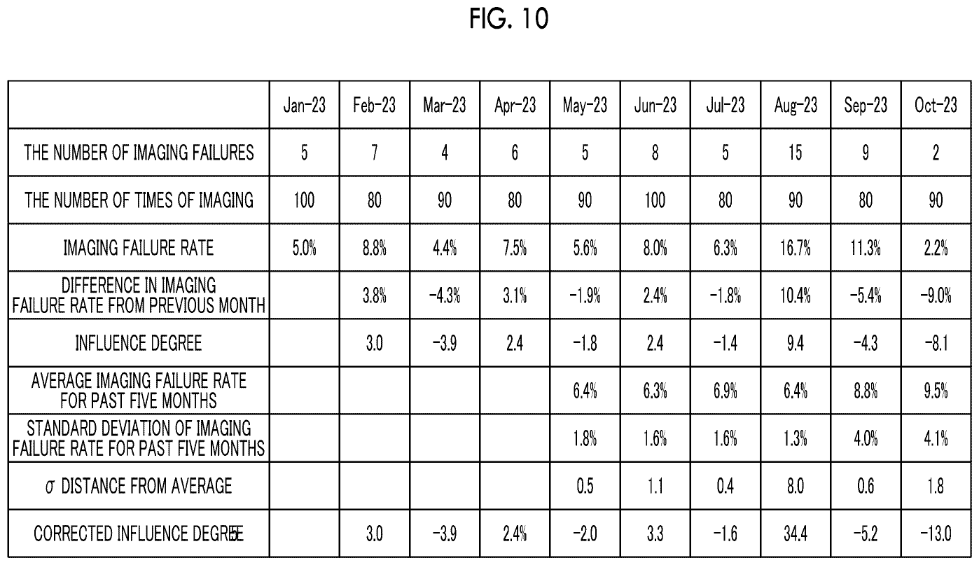

FIG. 10 is a diagram for describing the correction of the influence degree. FIG. 10 shows, as a table, the number of imaging failures, the number of times of imaging, the imaging failure rate, a difference in the imaging failure rate from the previous month, the influence degree, the average imaging failure rate for the past five months, the standard deviation of the imaging failure rate for the past five months, the σ distance from the average, and the corrected influence degree in a case in which each month from January to October 2023 (January-23 to October-23) is set as the reference target period T1 and the previous month is set as the comparison target period T2. The σ distance from the average is derived by dividing the imaging failure rate—an absolute value of the average imaging failure rate for the past five months by the imaging failure rate standard deviation for the past five months. In addition, the corrected influence degree is derived by the influence degree×(1+the σ distance from the average/3). In FIG. 10, in January to April in which there is no data for the past five months, the influence degree is not corrected.

In this case, the influence degree is corrected such that the absolute value of the influence degree becomes larger as the imaging failure rate derived in the reference target period T1 deviates from the average of the imaging failure rates for the past five months. As a result, the influence degree increases for menus with the same level of increase or decrease in the imaging failure rate and the number of times of imaging, as well as for menus with a higher imaging failure rate than usual. Therefore, it is possible to specify the menu having a different imaging failure rate than usual.

In addition, in the above-described embodiment, a variation in the influence degree for each item value of a second item different from a first item for which the influence degree is derived may be derived, and a display process for further displaying the variation may be performed. For example, the influence degree A0 may be derived for each imaging part as described above, and the variation in the influence degree for each technician may be derived for the influence degree for each imaging part. For the variation, for example, the standard deviation of the influence degree for each technician for each imaging part need only be used.

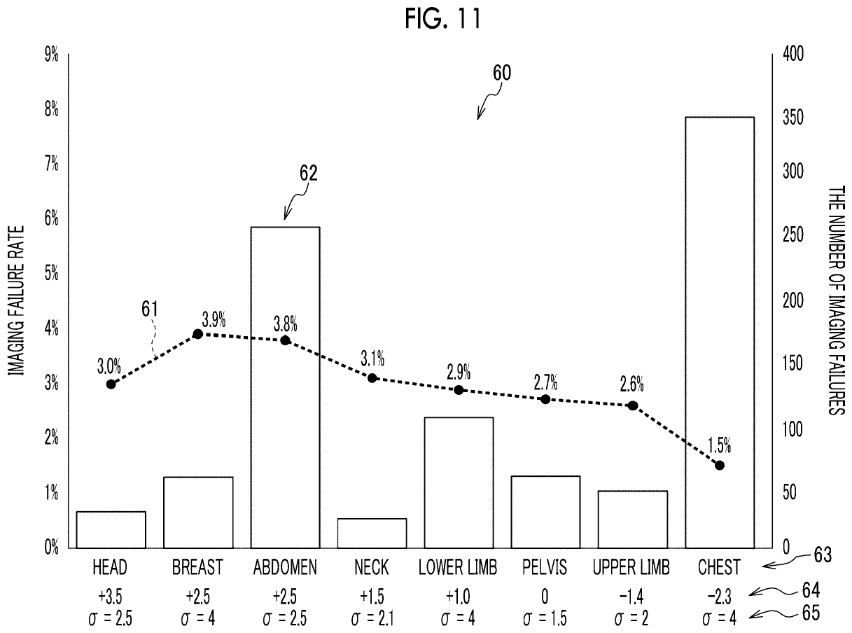

FIG. 11 is a diagram showing a display screen of the variation in the influence degree for each technician in a unit of an imaging part. FIG. 11 shows only the graph displayed in the main region 43 shown in FIG. 5. A graph 60 shown in FIG. 11 includes a line graph 61 of the imaging failure rate and a bar graph 62 of the number of imaging failures. A horizontal axis represents an imaging part, and FIG. 11 shows part names 63 of a head, a breast, an abdomen, a neck, a lower limb, a pelvis, an upper limb, and a chest. An influence degree 64 is shown below the part names 63, and a variation 65 in the influence degree for each technician for each part is shown as a value of the standard deviation σ below the influence degree 64.

As a result, it is possible to identify the variation in the influence degree for each technician with respect to the influence degree for each part, so that it is possible to use the result for training the technicians to improve their skills. For example, in a case in which the variation for each technician is large, it is considered that the number of imaging failures of a specific technician is large, so that it is possible to provide guidance to the technician from which the influence degree deviated from the average is derived. In a case in which the influence degree is large and the variation is small, it is considered that many technicians have made a large number of imaging failures for the part, so that it is possible to provide guidance to all the technicians.

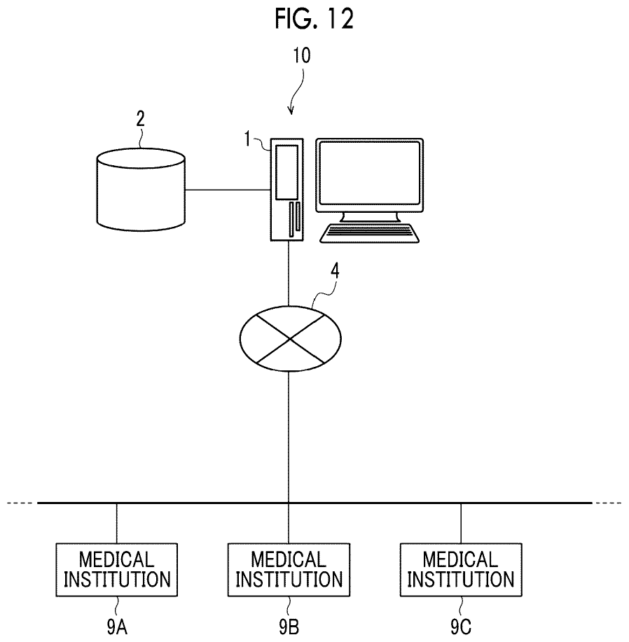

In addition, in the above-described embodiment, the difference is derived based on the medical practice information managed in the medical institution in a certain medical institution, and the influence degree is derived, but the present invention is not limited thereto. As shown in FIG. 12, the management server 2 may manage the medical practice information of a plurality of medical institutions 9A to 9C, and the medical service support device 1 may derive the influence degree on the increase or decrease in the index value of a certain medical institution (for example, the medical institution 9A) in consideration of the influence degree of the other medical institutions 9B and 9C.

In this case, the reception unit 21 receives, from the client terminal 3 of the medical institution 9A, an analysis instruction in consideration of the influence degree of the other medical institutions 9B and 9C. The information acquisition unit 22 acquires the medical practice information about a plurality of medical institutions 9B and 9C including the medical institution 9A from the management server 2. The first derivation unit 23 derives a difference (referred to as a first difference RS1) in the first statistical value for the medical institution 9A for each item value in each of the reference target period T1 and the comparison target period T2. In addition, the first derivation unit 23 derives a difference (referred to as a second difference RS2) in the first statistical value for a plurality of medical institutions 9B and 9C including the medical institution 9A for each item value in each of the reference target period T1 and the comparison target period T2.

The second derivation unit 24 derives the influence degree (referred to as a first influence degree A1) of the increase or decrease in the index value in the reference target period T1 for the medical institution 9A relative to the comparison target period T2 based on the first difference RS1 and the second statistical value for the medical institution 9A. In addition, the second derivation unit 24 derives the influence degree (referred to as a second influence degree A2) of the increase or decrease in the index value in the reference target period T1 relative to the comparison target period T2 based on the second difference RS2 and the second statistical value for the medical institution 9A. Both the first influence degree A1 and the second influence degree A2 may be derived, but only the second influence degree A2 may be derived.

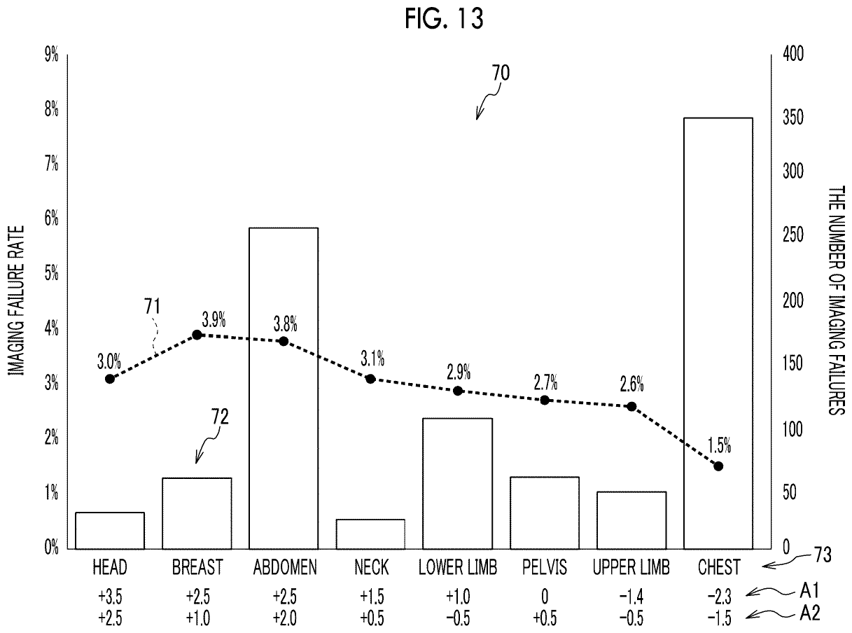

The display control unit 26 performs a process for displaying the first influence degree A1 and the second influence degree A2. FIG. 13 is a diagram showing a display screen of an influence degree in consideration of influence degrees of a plurality of medical institutions. FIG. 13 shows only the graph displayed in the main region 43 shown in FIG. 5. A graph 70 shown in FIG. 13 includes a line graph 71 of the imaging failure rate and a bar graph 72 of the number of imaging failures in the medical institution 9A. A horizontal axis represents an imaging part, and FIG. 13 shows part names 73 of a head, a breast, an abdomen, a neck, a lower limb, a pelvis, an upper limb, and a chest, as in FIG. 11. The first influence degree A1 for the medical institution 9A is shown below the part names 73, and the second influence degree A2 derived for the medical institutions 9A to 9C is displayed below the first influence degree A1.

By deriving the second influence degree A2 in this way, the medical institution that has requested the analysis can obtain an analysis result of the increase or decrease in the index value of the medical institution itself in consideration of the increase or decrease in the index value in the other medical institutions.

In addition, in the above-described embodiment, the influence degree may be displayed by a graph such as a line graph or a bar graph.

In addition, in the above-described embodiment, the imaging failure rate is used as the first statistical value, but the present invention is not limited thereto. For example, the reason for re-imaging included in the medical practice information shown in FIG. 2 may be used as the item value, and the number of imaging failures for each reason for re-imaging in each of the reference target period T1 and the comparison target period T2 may be used as the first statistical value. In this case, the number of imaging failures need only be derived by counting the number of imaging failures in each of the reference target period T1 and the comparison target period T2.

In addition, in the above-described embodiment, the reception unit 21 receives the designation of the reference target period T1 and the comparison target period T2, but the present invention is not limited thereto. Analysis for a predetermined reference target period T1 and a predetermined comparison target period T2 may be performed without receiving the designation of the reference target period T1 and the comparison target period T2.

In the above-described embodiment, for example, as a hardware structure of processing units that execute various types of processing, such as the reception unit 21, the information acquisition unit 22, the first derivation unit 23, the second derivation unit 24, the notification unit 25, and the display control unit 26, various processors shown below can be used. As described above, the various processors include, in addition to a CPU which is a general-purpose processor that executes software (program) to function as various processing units, a graphics processing unit (GPU), a programmable logic device (PLD) which is a processor whose circuit configuration can be changed after manufacturing such as a field programmable gate array (FPGA), and a dedicated circuitry which is a processor having a circuit configuration specifically designed to execute specific processing such as an application specific integrated circuit (ASIC).

One processing unit may be configured of one of the various types of processors, or a combination of two or more processors of the same type or different types (for example, a combination of a plurality of FPGAs, or a combination of a CPU and an FPGA). In addition, a plurality of processing units may be configured of one processor.

As an example of configuring a plurality of processing units with one processor, first, there is a form in which, as typified by computers such as a client and a server, one processor is configured by combining one or more CPUs and software, and the processor functions as a plurality of processing units. Second, there is a form in which, as typified by a system on chip (SoC) and the like, in which a processor that implements functions of an entire system including a plurality of processing units with one integrated circuit (IC) chip is used. As described above, various processing units are configured by using one or more of the various processors as a hardware structure.

Furthermore, as the hardware structure of the various types of processors, more specifically, an electric circuit (circuitry) in which circuit elements such as semiconductor elements are combined can be used.

Appendices of the present disclosure will be described below.

### Appendix 1

A medical service support device comprising:
at least one processor,
in which the processor is configured to
   acquire medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value,
   derive a difference between a first statistical value of the information related to the index value in the comparison target period and a first statistical value of the information related to the index value in the reference target period for each of a plurality of item values of at least one predetermined item,
   derive an influence degree on the increase or decrease in the index value in the reference target period relative to the comparison target period for each item value of the at least one item, based on a second statistical value based on the medical practice information in the reference target period and the difference, and
   perform a display process for displaying the influence degree for at least one item value included in the at least one item.

### Appendix 2

The medical service support device according to Appendix 1,
in which the medical practice includes at least one of an interview, an examination, a treatment, or a nursing care.

### Appendix 3

The medical service support device according to Appendix 1 or 2,
in which the index value includes at least one of presence or absence of a failure of the medical practice, a time related to the medical practice, an index value of a radiation dose generated by the medical practice, or an index value based on an image acquired by the medical practice.

### Appendix 4

The medical service support device according to any one of Appendices 1 to 3,
in which the date and time related to the medical practice include at least one of a start date and time of the medical practice, an end date and time of the medical practice, a reservation date and time of the medical practice, a reception date and time of the medical practice, or a call date and time for implementation of the medical practice.

### Appendix 5

The medical service support device according to any one of Appendices 1 to 4,
in which the processor is configured to perform a display process for displaying the influence degree for each item value in ascending order or descending order.

### Appendix 6

The medical service support device according to Appendix 5,
in which the processor is configured to perform a display process for displaying the influence degree for a predetermined number of item values with a large or small influence degree in a manner distinguished from the influence degree for other item values.

### Appendix 7

The medical service support device according to any one of Appendices 1 to 6,
in which the processor is configured to
   receive designation of a second item different from a first item for which the influence degree is derived, and
   perform a display process for displaying the plurality of item values in an order corresponding to the influence degree for at least one item value included in the second item.

### Appendix 8

The medical service support device according to any one of Appendices 1 to 7,
in which the processor is configured to perform a display process for displaying the first statistical value in the reference target period for each item value, in addition to the influence degree.

### Appendix 9

The medical service support device according to Appendix 8,
in which the processor is configured to perform a display process for displaying at least one of the influence degree, the first statistical value, or the second statistical value in a graph.

### Appendix 10

The medical service support device according to any one of Appendices 1 to 9,
in which the processor is configured to
   derive the difference for each of the plurality of item values for each of a plurality of items,
   derive the influence degree for each of the plurality of item values for each of the plurality of items, and
   perform a display process for displaying the influence degree for at least one item value included in each of the plurality of items, for each of the plurality of items.

### Appendix 11

The medical service support device according to any one of Appendices 1 to 10,
in which the processor is configured to correct the influence degree based on a distribution of the first statistical value in a specific period longer than the reference target period or the comparison target period, and perform a display process for displaying the corrected influence degree.

### Appendix 12

The medical service support device according to any one of Appendices 1 to 11,
in which the processor is configured to notify an external device of the derivation of the influence degree.

### Appendix 13

The medical service support device according to Appendix 12,
in which the processor is configured to, in a case in which the derived influence degree satisfies a predetermined condition, notify of the influence degree.

### Appendix 14

The medical service support device according to any one of Appendices 1 to 13,
in which the processor is configured to
   derive a variation in the influence degree for each item value of a second item different from a first item for which the influence degree is derived, and
   perform a display process for further displaying the variation.

### Appendix 15

The medical service support device according to any one of Appendices 1 to 14,
in which the processor is configured to
   derive the difference as a first difference based on the medical practice information for a first medical institution among a plurality of medical institutions,
   derive a difference between a first statistical value of the information related to the index value in the comparison target period and a first statistical value of the information related to the index value in the reference target period as a second difference based on the medical practice information for the plurality of medical institutions in the comparison target period,
   instead of or in addition to the influence degree, derive another influence degree on the increase or decrease in the index value in the reference target period relative to the reference target period for each item value of the at least one item based on the first difference, the second difference, and the second statistical value for the first medical institution, and
   perform a display process for displaying, instead of or in addition to the influence degree, the other influence degree.

### Appendix 16

The medical service support device according to any one of Appendices 1 to 15,
in which the processor is configured to receive designation of the reference target period and the comparison target period.

### Appendix 17

A medical service support method comprising:
acquiring medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value;
deriving a difference between a first statistical value of the information related to the index value in the comparison target period and a first statistical value of the information related to the index value in the reference target period for each of a plurality of item values of at least one predetermined item;
deriving an influence degree on the increase or decrease in the index value in the reference target period relative to the comparison target period for each item value of the at least one item, based on a second statistical value based on the medical practice information in the reference target period and the difference; and
performing a display process for displaying the influence degree for at least one item value included in the at least one item.

### Appendix 18

A medical service support program causing a computer to execute:
a procedure of acquiring medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value;
a procedure of deriving a difference between a first statistical value of the information related to the index value in the comparison target period and a first statistical value of the information related to the index value in the reference target period for each of a plurality of item values of at least one predetermined item;
a procedure of deriving an influence degree on the increase or decrease in the index value in the reference target period relative to the comparison target period for each item value of the at least one item, based on a second statistical value based on the medical practice information in the reference target period and the difference; and
a procedure of performing a display process for displaying the influence degree for at least one item value included in the at least one item.

What is claimed is:

1. A medical service support device comprising:
at least one processor,
wherein the processor is configured to
acquire medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value,
derive a difference between a first statistical value of the information related to the index value in the comparison target period and a first statistical value of the information related to the index value in the reference target period for each of a plurality of item values of at least one predetermined item,
derive an influence degree on the increase or decrease in the index value in the reference target period relative to the comparison target period for each item value of the at least one item, based on a second statistical value based on the medical practice information in the reference target period and the difference, and
perform a display process for displaying the influence degree for at least one item value included in the at least one item.

2. The medical service support device according to claim 1,
wherein the medical practice includes at least one of an interview, an examination, a treatment, or a nursing care.

3. The medical service support device according to claim 1,
wherein the index value includes at least one of presence or absence of a failure of the medical practice, a time related to the medical practice, an index value of a radiation dose generated by the medical practice, or an index value based on an image acquired by the medical practice.

4. The medical service support device according to claim 1,
wherein the date and time related to the medical practice include at least one of a start date and time of the medical practice, an end date and time of the medical practice, a reservation date and time of the medical practice, a reception date and time of the medical practice, or a call date and time for implementation of the medical practice.

5. The medical service support device according to claim 1,
wherein the processor is configured to perform a display process for displaying the influence degree for each item value in ascending order or descending order.

6. The medical service support device according to claim 5,
wherein the processor is configured to perform a display process for displaying the influence degree for a predetermined number of item values with a large or small influence degree in a manner distinguished from the influence degree for other item values.

7. The medical service support device according to claim 1,
wherein the processor is configured to
receive designation of a second item different from a first item for which the influence degree is derived, and
perform a display process for displaying the plurality of item values in an order corresponding to the influence degree for at least one item value included in the second item.

8. The medical service support device according to claim 1,
wherein the processor is configured to perform a display process for displaying the first statistical value in the reference target period for each item value, in addition to the influence degree.

9. The medical service support device according to claim 8,
wherein the processor is configured to perform a display process for displaying at least one of the influence degree, the first statistical value, or the second statistical value in a graph.

10. The medical service support device according to claim 1,
wherein the processor is configured to
derive the difference for each of the plurality of item values for each of a plurality of items,
derive the influence degree for each of the plurality of item values for each of the plurality of items, and
perform a display process for displaying the influence degree for at least one item value included in each of the plurality of items, for each of the plurality of items.

11. The medical service support device according to claim 1,
wherein the processor is configured to correct the influence degree based on a distribution of the first statistical value in a specific period longer than the reference target period or the comparison target period, and perform a display process for displaying the corrected influence degree.

12. The medical service support device according to claim 1,
wherein the processor is configured to notify an external device of the derivation of the influence degree.

13. The medical service support device according to claim 12,
wherein the processor is configured to, in a case in which the derived influence degree satisfies a predetermined condition, notify of the influence degree.

14. The medical service support device according to claim 1,
wherein the processor is configured to
derive a variation in the influence degree for each item value of a second item different from a first item for which the influence degree is derived, and
perform a display process for further displaying the variation.

15. The medical service support device according to claim 1,
wherein the processor is configured to
derive the difference as a first difference based on the medical practice information for a first medical institution among a plurality of medical institutions,
derive a difference between a first statistical value of the information related to the index value in the comparison target period and a first statistical value of the information related to the index value in the reference target period as a second difference based on the medical practice information for the plurality of medical institutions in the comparison target period,
instead of or in addition to the influence degree, derive another influence degree on the increase or decrease in the index value in the reference target period relative to the reference target period for each item value of the at least one item based on the first difference, the second difference, and the second statistical value for the first medical institution, and
perform a display process for displaying, instead of or in addition to the influence degree, the other influence degree.

16. The medical service support device according to claim 1,
wherein the processor is configured to receive designation of the reference target period and the comparison target period.

17. A medical service support method comprising:
acquiring medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value;
deriving a difference between a first statistical value of the information related to the index value in the comparison target period and a first statistical value of the information related to the index value in the reference target period for each of a plurality of item values of at least one predetermined item;
deriving an influence degree on the increase or decrease in the index value in the reference target period relative to the comparison target period for each item value of the at least one item, based on a second statistical value based on the medical practice information in the reference target period and the difference; and
performing a display process for displaying the influence degree for at least one item value included in the at least one item.

18. A non-transitory computer-readable storage medium that stores a medical service support program causing a computer to execute:
a procedure of acquiring medical practice information including a date and time related to a medical practice and information related to an index value related to the medical practice, the date and time being included in a reference target period, which is a target period for analysis of an increase or decrease in the index value, and a comparison target period, which is a target period for comparison of the increase or decrease in the index value;
a procedure of deriving a difference between a first statistical value of the information related to the index value in the comparison target period and a first statistical value of the information related to the index value in the reference target period for each of a plurality of item values of at least one predetermined item;
a procedure of deriving an influence degree on the increase or decrease in the index value in the reference target period relative to the comparison target period for each item value of the at least one item, based on a second statistical value based on the medical practice information in the reference target period and the difference; and
a procedure of performing a display process for displaying the influence degree for at least one item value included in the at least one item.
